(19) United States
(12) Patent Application Publication
(10) Pub. No.: US 2021/0100955 A1
(43) Pub. Date: Apr. 8, 2021

(54) SPRING FOR AN INJECTION DEVICE

(71) Applicant: Ypsomed AG, Burgdorf (CH)

(72) Inventors: Peter Stettler, Ersign (CH); Benjamin Loretz, Riedholz (CH); Lorenzo Notaro, Bern (CH)

(21) Appl. No.: 17/125,946

(22) Filed: Dec. 17, 2020

Related U.S. Application Data
(63) Continuation of application No. PCT/IB2019/053762, filed on May 8, 2019.

(30) Foreign Application Priority Data
Jun. 19, 2018 (CH) 00773/18

(51) Int. Cl. A61M 5/20; A61M 5/32; F16F 1/06

(52) U.S. Cl. CPC A61M 5/2033; A61M 5/3243; F16F 1/06; F16F 2238/026; F16F 2226/048; F16F 2228/007; A61M 2207/00

(57) ABSTRACT

An injection device for administering a medical substance includes a helical spring having a coil that extends in an axial direction and has at least three windings, where two adjacent windings are non-detachably connected to one another at a point in the axial direction by means of a laser-welded connection. The helical spring and a method for producing the helical spring for use with the injection device are also provided.

### CROSS-REFERENCE TO RELATED APPLICATIONS

This application claims priority to International Patent Application No. PCT/IB2019/053762, filed May 8, 2019, entitled “SPRING FOR AN INJECTION DEVICE,” which in turn claims priority to Swiss Patent Application No. 00773/18, filed Jun. 19, 2018, entitled “SPRING FOR AN INJECTION DEVICE”, each of which is incorporated by reference herein, in the entirety and for all purposes.

### TECHNICAL FIELD

The device and method in question concern the field of medical injection devices for administering liquid substances, in particular medicines or medical substances such as insulin and hormone formulations. The disclosure relates to an injection device with a coiled spring and a procedure for producing the coiled spring.

### BACKGROUND

At present in this field, we are aware of various different types of injection devices for administering liquid medical substances. Injection devices known as autoinjectors allow the automatic release of the medical substance through a pre-loaded release spring, which squeezes a stopper into a syringe using a piston rod. Such autoinjectors usually feature a protective shield or sleeve for the needle, which can slide by means of a coiled spring longitudinally between a proximal and distal position relative to the casing or housing of the autoinjector. After the injection, the pre-loaded coiled spring pushes the protective sleeve into the distal position, in which it surrounds and shields the needle from the sides. The protective sleeve thus ensures that the user is not injured by the needle after the injection.

With conventional coiled springs extending and acting axially, a spring end or the end of the coil of the coiled spring can sometimes be displaced radially inwards or outwards, or, if too much stress is applied to the coiled spring, a spring end may shift radially inwards or outwards. In this case, the loops of the coil do not all remain aligned, one above the other, during an axial compression or expansion of the coiled spring. As such, the coiled spring does not compress or expand fully, or not quite in the correct axial direction. There is a risk that individual loops may get caught on one another, preventing the coiled spring from compressing and expanding fully or as well as it should.

It is true that the spring can be guided by the wall of the casing if it sits tightly against the coiled spring in such a way that a radial deflection of the spring end is not possible. However, such a construction is complex and also makes the installation of the coiled spring more difficult. Moreover, due to the contact of the coil with the wall of the casing, unwanted losses to friction may occur.

In addition, one option would be to automatically pick out the coiled springs where the spring end of the coil is displaced radially inwards or outwards by means of optical detection before they are installed. A system with such an optical detection function is very expensive, however, and makes the installation process complex.

In other technical areas, such as in the field of spiral springs, such problems cannot arise, since the spiral springs are generally made of a rectangular strip material. Due to their shape, the spring ends do not tend to shift. As a result, the spring ends of spiral coils can even be used for mounting support. Such a use is described for example in WO 2011/081586 A1. This disclosure presents a spiral spring designed to hold a material in a coiled position on a roller. The spiral coil is made up of several loops of which the two outermost loops are connected with one another in a radial direction to form an integrated retainer, which prevents the spring material from releasing back into its original position. The loops are attached to one another by means of laser welding.

Spiral springs may be used within injection devices as torsion springs for the propulsion of the release. However, for linear spring-loaded elements in the injection device, such as the aforementioned protective sleeve, tension springs or compressible coiled springs are preferred.

### SUMMARY

The purpose of the invention is therefore to enable a safe and reliable shifting of the protective sleeve within the injection device.

The task is performed by an injection device with a coiled spring and by a coiled spring with two loops connected to one another, in accordance with the claims. Various alternative designs are also in the claims.

In accordance with the disclosure, the injection device contains a coiled spring which extends as a coil in an axial direction with at least three loops, of which two adjacent loops are fixedly, non-detachably or inextricably joined to one another in the axial direction at one point by means of welding, including laser welding.

In this way, the loops which are joined to one another cannot shift radially relative to one another upon compression or expansion of the coiled spring. The coiled spring thus retains its original exterior dimensions when subjected to stress. In this way, the coiled spring can compress and expand properly and fully in the axial direction. Moreover, there is no risk that the coils will get caught on one another causing the coiled spring to no longer have the capacity to become fully tensioned or un-tensioned. The elements within the injection device, which work together with the coiled spring, such as the needle's protective sleeve, can thus be moved by the coiled spring properly. There is therefore no need for a costly and time-consuming removal of coiled springs in which the spring ends have shifted radially inwards or outwards.

Furthermore, the invention also relates to the procedure for joining two loops of a coiled spring for the purpose of an injection device. The process includes the following steps: provision of a coiled spring, which extends as a coil in an axial direction with at least three loops, and the joining of two adjacent loops at one point by welding, including laser welding.

Laser welding at a point allows the loops to be non-detachably joined to one another swiftly and reliably in an axial direction. The process of joining the two adjacent loops can thus occur in less than five seconds in an automated production factory. In this way, large quantities of these coiled springs can be produced efficiently.

According to the present disclosure, the term “medicine” or “medical substance” includes any liquid medical formulation that is subject to controlled administration through a cannula or hollow needle. For example, this may include a liquid, a solution, a gel, or a fine suspension containing one or more medical agents. A medicine can thus be a composition with a single agent or a pre-mixed or co-formulated composition with several agents out of a single container. In particular, the term encompasses drugs such as peptides (e.g., insulins, medicines containing insulin or containing GLP-1 as well as other formulae derived from these), proteins and hormones, biological or active agents, agents derived from hormones or genes, nutritional formulations, enzymes and other substances in either solid (suspended) or liquid state. The term also includes polysaccharides, vaccines, DNA or RNA or oligonucleotides, antibodies or parts of antibodies, as well as appropriate raw materials, auxiliary agents and excipients.

The term “injection device” or “injector” is used herein to refer to a device in which the injection needle is removed from the tissue of the patient after the release of the medical substance. Thus, in contrast to an infusion system, the injection needle of an injection device or injector does not stay in the patient permanently or for an extended period of several hours.

A coiled spring is made up of a coil extending in an axial direction with several loops. The coiled spring is thus formed by a helically-coiled, elongated material, hereinafter referred to as the wire. In this instance, it is not necessary for the wire itself to have a circular cross section. The wire cross section can function just as well in a rectangular, polygon or elliptical shape. The coiled spring can be compressed or expanded along its longitudinal axis or in an axial direction. The coiled spring is differentiated fundamentally in this way from a spiral spring. The latter is formed by a strip coiled around a center-point on a single flat plane (e.g., configured as a flat spiral to store and release rotational energy), where the spiral spring can be tensed or de-tensed by rotating one of its ends.

The term “loop” refers to a segment (or turn or winding) of the coil, which extends around a 360° angle or less. A loop begins where a previous loop has finished its 360° extension or where the elongated material (wire) begins. Hence, a loop ends after it has finished its 360° extension or where the elongated material (wire) ends. According to this definition, the two outermost loops of the coil of a coiled spring (at the ends of the coiled spring) extend through an angle of exactly 360° or less and the inner loops, which are between the two outermost loops, each extend through an angle of 360°.

The term “distal” is used here to refer to the direction facing the end of the injection device with the needle. On the other hand, the term “proximal” refers to the direction facing the rear end of the injection device, opposite the needle end.

The injection device is ideally made up of a casing or housing and a protective sleeve, which can be moved by the coiled spring in a distal direction relative to the casing. As a result, the coiled spring should be tensionable or loadable by a movement of the protective sleeve. The coiled spring can be a compression spring (where the coiled spring is tensed or loaded by compression) or a tension spring (where the coiled spring is tensed or loaded by expansion).

Ideally, the coiled spring should be tensed when the protective sleeve is in a proximal position, whereby the point of the needle is not covered by the protective sleeve. However, when the protective sleeve is in a distal position, whereby the point of the needle is covered by the protective sleeve, then the coiled spring is relaxed or less tensed. In this way, the risk of injury by a needle is significantly reduced.

Alternatively, it is also possible for the coiled spring to move other elements of the injection device, for example, the drive mechanism for releasing the medical substance out of the syringe or a moveable display or adjusting element.

Preferably, the injection device should include a spring retaining space with an annular cross section in which the coiled spring is at least partly surrounded or held. The annular cross section can also be described as a volume or a spring chamber bounded by two concentrically, arranged, circular, cylindrical surfaces. The annular cross section of the spring retaining space should have a cross-sectional width of the space which is, at least in sections, greater than 1.3 times the external dimension of the wire of the coiled spring.

That means that the coiled spring is not tight within the spring retaining space; therefore, the loops of the coiled spring do not, or only barely, touch the cylindrical walls, which border or define the spring retaining space. In this way, the friction and thus the mechanical wear between the coiled spring and the surrounding walls is reduced. In addition, the process of producing the elements that form the spring retaining space is simplified, as it avoids the need to adhere to tight tolerances. Furthermore, the spring retaining space with its wide annular cross-section makes the installation of the coiled spring easier.

Moreover, the invention relates to a coiled spring for an injection device, as described above, in which a coiled spring extends in an axial direction with at least three loops, of which two adjacent loops are fixedly joined to one another at one point in the axial direction through laser welding.

In one embodiment an outermost and an adjacent or second outermost loop of the coil are joined to one another through laser welding, because the spring end or the end of the wire of the coiled spring is most likely to be displaced radially inwards or outwards on compression or expansion of the coiled spring. The spring ends can thus be stabilized so that the coiled spring can compress and expand correctly and fully in an axial direction.

Alternatively, it is also possible that two of the inner loops distinct from the outermost or end loops—are joined to one another, for example the second and third outermost loops.

In addition, the outermost and second outermost loops at each end of the coiled spring respectively are preferably connected to one another. In this way, the coiled spring is symmetrical, which makes it easier to use.

In an embodiment, the loops will be formed from a wire with a circular cross section. Such a wire is readily available and the coiled spring is particularly easily wound with a wire that has a circular cross section. In an embodiment, the wire is made of stainless spring steel and is also electro-conductive. Thanks to its electrical conductivity, the position of the spring can be detected, if required, with an inductive or capacitive sensor, for example.

Alternatively, the coiled spring can also be formed from a rectangular cross-section wire, for example. This is more complex to produce, however, due to the required alignment. In addition, the raw material for a coiled spring made of rectangular wire is difficult to procure.

Preferably, the diameter of the wire will be smaller than 2 mm and specifically between 0.3 mm and 1.2 mm. Furthermore, the coiled spring in one embodiment may be configured as a compression spring with a spring constant of less than 0.2 N/mm.

The spring's two loops joined may in one embodiment be joined to one another at two separate points by means of laser welding. The two loops are thus joined to one another in a particularly stable state. In addition, it also ensures that even if one of the two laser-welded connections breaks, the two loops are still joined to one another.

The coiled spring may consist of at least 5 loops, in various embodiments between 10 and 30 loops. Moreover, the spring may be a length of less than 200 mm in an unstressed (or un-tensed or unloaded) position, i.e., when neither compressed nor expanded.

The external dimensions of the coiled spring in one embodiment should be between 4 mm and 26 mm and in some embodiments between 9 mm and 21 mm. In one embodiment, the external dimension (or diameter) of the coiled spring which is perpendicular to the axis should be at least 9 mm and the connection of loops is positioned at a maximum of 20 mm from one end of the wire of the coiled spring, preferably a maximum of 5 mm, measured in a direction along a coil circumferential. In this way, the end of the wire is fixed optimally so that it cannot shift radially inwards or outwards.

### BRIEF DESCRIPTION OF THE DRAWINGS

One design or embodiment of the invention will now be laid out in association with the figures. These demonstrate fundamental possibilities of the design and are in no way meant to be restrictive.

FIG. 1 shows a perspective image of a coiled spring according to this disclosure.

FIG. 2a shows a cross section view taken along the longitudinal axis of an exemplary injection device with the coiled spring in a de-tensed or unloaded position.

FIG. 2b shows a cross section view of the injection device rotated 90° from FIG. 2a, around the longitudinal axis.

FIG. 3a shows a cross section view of the injection device with the coiled spring in a compressed position.

FIG. 3b shows a cross section view of the injection device rotated 90° from FIG. 3a, around the longitudinal axis.

### DETAILED DESCRIPTION

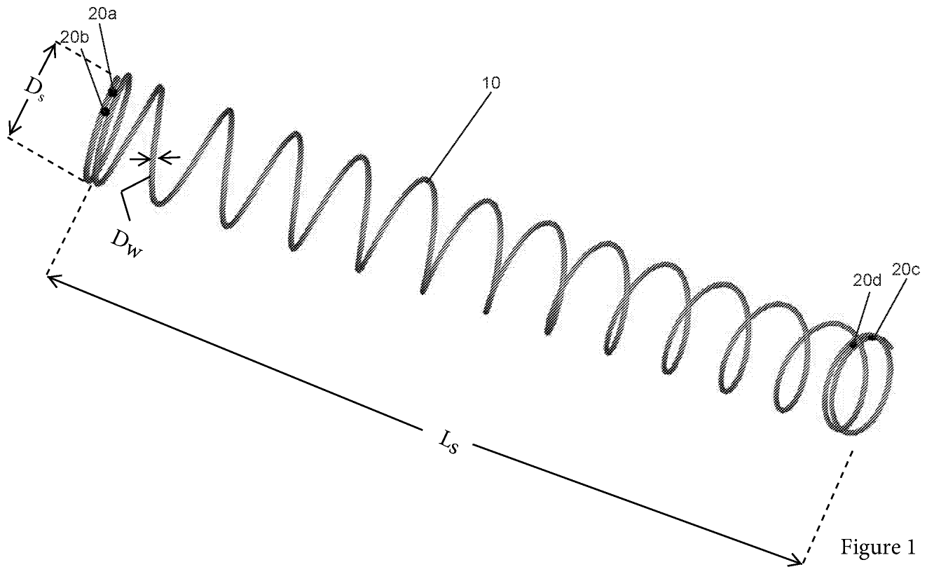

FIG. 1 is a perspective, overview image of a coiled spring 10 embodying the disclosure. The coiled spring 10 is set up as a compression spring and consists of a coil (or windings) that extends in an axial direction or in a longitudinal direction of the coiled spring 10 with 15 loops in total. In this example, the coiled spring 10 has a length Ls of 114 mm in an un-tensed position. It also has an external diameter D_{S} of 15 mm. The coiled spring 10 is made of a wire with a circular cross section with a diameter D_{W} of 0.7 mm. The wire is made of spring steel with a chrome-nickel alloy and is also electro-conductive. As can be seen in FIG. 1, the two outermost loops or windings on each end are connected to one another in at least one point by means of laser spot welding.

The outermost or first loop and the second outermost or second loop of the coil 10 are joined to one another at two connection points 20a, 20b, at one end, 20c, 20d, at the other. A first point of connection 20a is around 2 mm away from one of the ends of the wire, but this can be greater if desired. A second point of connection 20b is 3 mm from the same end of the wire or 1 mm from the first point of connection.

This arrangement of the points of connection 20a, 20b at the first end of the coiled spring has a corresponding arrangement in a third and a fourth point of connection 20c, 20d on the other end of the coiled spring 10. The coiled spring 10 thus has a total of four connection points 20a, 20b, 20c, 20d or welded spots.

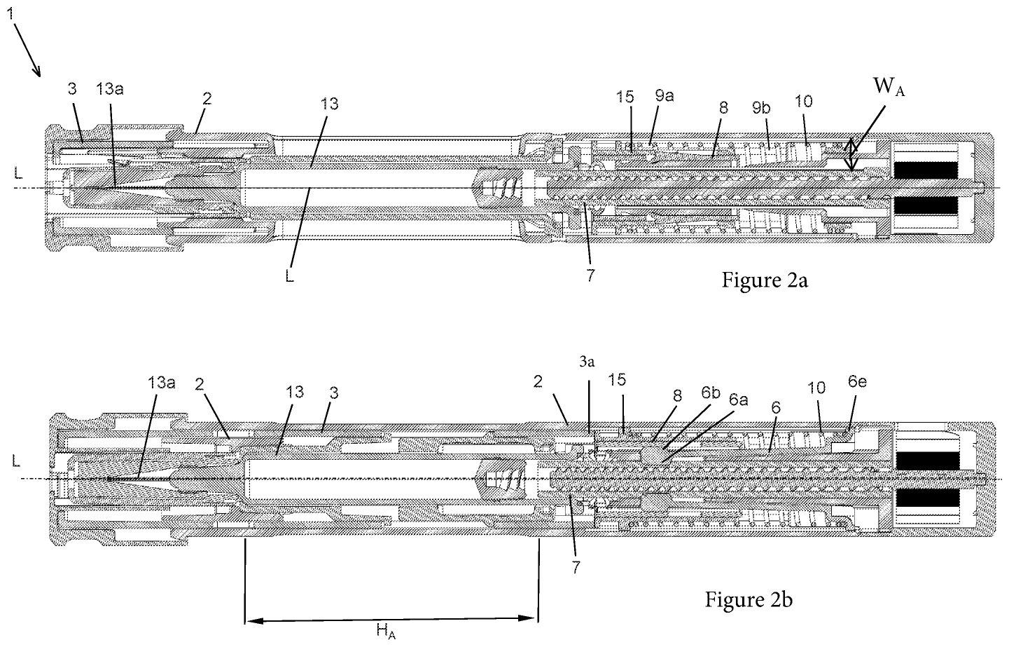

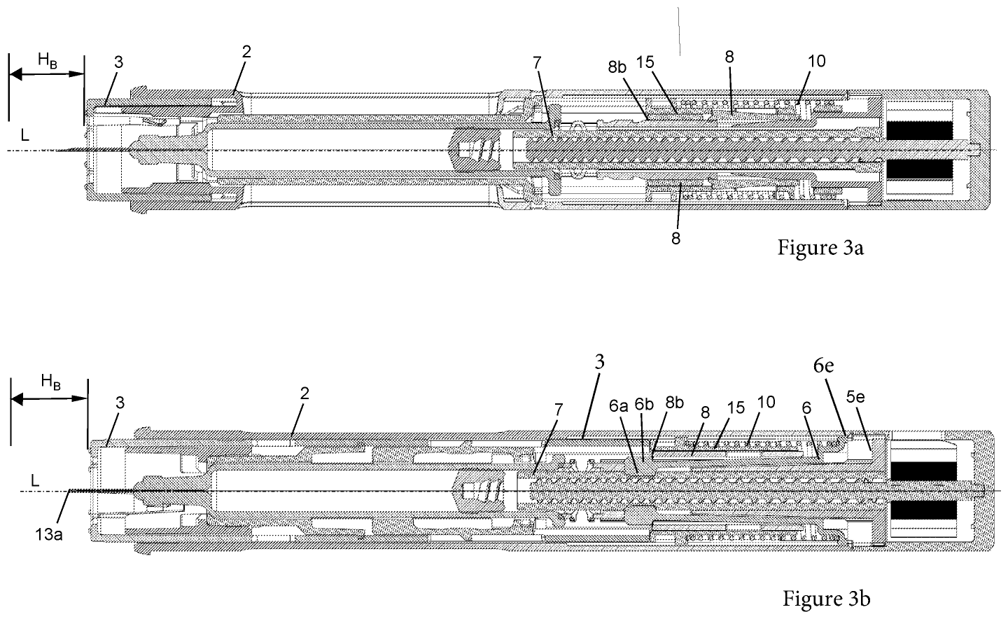

FIGS. 2a, 2b, 3a and 3b each show a cross section of an autoinjector 1 with the coiled spring 10 according to the present disclosure. In each case, the cross section view is taken along the longitudinal axis L of the autoinjector 1. The cross section in FIGS. 2b and 3h reflects the views of FIGS. 2a and 3a, respectively, rotated 90° around the longitudinal axis L. In the following, the arrangement and function of the coiled spring 10 within the autoinjector 1 will be laid out with reference to these figures.

The autoinjector 1 comprises a sleeve-like elongated casing or housing 2 with a longitudinal axis L. In the casing 2, there may be a product container 13 with the medicine. On the distal end, the product container 13 is joined to a hollow injection needle 13a. The autoinjector 1 also has a protective sleeve 3 that is movable in a proximal direction relative to the casing 2 and along the longitudinal axis L during the actuator stroke (H_{B}) to the activated position in order to trigger the release of the product.

In the casing 2, there is also a trigger sleeve 15 and a locking sleeve 8. The trigger sleeve 15 is positioned at a proximal end 3a of the protective sleeve 3. The coiled spring 10 is oriented in a longitudinal direction within the autoinjector and partly surrounds both the trigger sleeve 15 and the locking sleeve 8. The coiled spring 10 partially supports itself with a circumferential portion of its distal end, seen from a circumferential direction resting on the trigger sleeve 15. Part of the trigger sleeve 15 is thus positioned between the protective sleeve 3 and the distal end of the coiled spring 10. In this way, the coiled spring 10 can be seen in FIG. 2a with its distal portion in an annular volume 9a which is formed by the exterior surface of the trigger sleeve 15 and an inner wall of the casing 2. In addition, the coiled spring 10 is supported at its proximal end on a projection 6e of a retainer element 6, as is shown in FIG. 2b. The retainer element 6 protrudes into the casing 2 and can slide axially without rotating. In its proximal portion, the coiled spring 10 is also arranged in an annular volume 9b, which is formed by the exterior surface of the retainer element 6 and the inner wall of the casing 2. The coiled spring 10 is thus only supported at its distal and proximal ends. The annular volumes 9a, 9b are significantly wider than the diameter of the wire D_{W} of the coiled spring 10. In sections, the width WA of the annular volume 9a, 9b is approximately, twice as wide as the diameter D_{W} of the wire. As such, the loops of the coiled spring 10 hardly, touch the walls of the annular volume 9a, 9b between the two ends of the coiled spring 10.

In the starting position of the protective sleeve 3, as shown in FIGS. 2a and 2b, the distal end of the protective sleeve 3 protrudes distally beyond the needle point of the injection needle 13a, initially preventing the needle point from being contacted, and therefor may be referred to as a needle protective sleeve.

In order to administer the medicine out of the product container 13, the distal end of the protective sleeve 3 is positioned on a patient's intended point of injection, the casing 2 is pushed toward the point of injection and the protective sleeve 3 is displaced from its starting position in the proximal direction relative to the casing 2, via the actuator stroke (HO. As a result of this, the coiled spring 10 is compressed and thus tensed, whereby the trigger sleeve 15 and the locking sleeve 8 are displaced together with the protective sleeve 3 via the actuator stroke (He). This state (with stroke He) is depicted in FIGS. 3a and 3b.

As a result of the displacement of the locking sleeve 8, e.g., in the proximal direction, a piston driving member 7 is released via a protrusion 6a, causing a movement in the distal direction along the release stroke (HA), by which the release of the medicine out of the product container 13 begins.

At the end of the release stroke (HA), the protrusions fib of the retainer element 6 are moved out of a recess 8b of the locking sleeve 8. Thus, the tensed coiled spring 10 can relax in part and then the retainer element 6 can accelerate in the proximal direction, as a result of which an acoustic and/or tactile signal is produced when the retainer element 6 meets an end signal stop 5e.

When the autoinjector 1 is removed from the injection point, the protective sleeve 3 is free to move back into a distal position; the coiled spring 10 relaxes and thus moves the trigger sleeve 15 and the protective sleeve 3 out of the actuation position into the protective position via the protective sleeve return stroke (distally along H_{B} shown in the figures).

### KEY FOR REFERENCE NUMBERS

1 Autoinjector
2 Casing
3 Needle protective sleeve
3a Proximal end of sleeve 3
5e Final signal stop
6 Retainer element
6a Protrusion
6b Protrusion
6e Retainer Projection
7 Driving member
8 Locking sleeve
8b Recess
9a Annular volume (proximal)
9b Annular volume (distal)
10 Coiled spring
13 Product container
13a Hypodermic needle
15 Trigger sleeve
20a First connection point
20b Second connection point
20c Third connection point
20d Fourth connection point

What is claimed is:

1. An injection device for administering a medical substance, comprising a coiled spring which extends in an axial direction as a coil with at least three loops, of which two adjacent loops are fixedly joined in the axial direction to one another in at least one point by means of a welded connection.

2. The injection device of claim 1, further comprising a casing and a protective sleeve for needle protection, wherein the protective sleeve is movable by the coiled spring in a distal direction in relation to the casing.

3. The injection device of claim 1, further comprising a spring retaining volume with an annular cross section surrounding at least part of the coiled spring, wherein the annular cross section of the spring retaining volume has a cross-sectional width of more than 1.3 times an external diameter dimension of a wire in the coiled spring, at least in sections.

4. A coiled spring for an injection device for administering a medical substance, wherein the coiled spring extends as a coil in an axial direction with at least three loops, of which two adjacent loops are fixedly joined to one another in at least one point in the axial direction by means of a welded connection.

5. The coiled spring of claim 4, wherein an outermost loop and a second from outermost loop of the coil are joined to one another by the welded connection.

6. The coiled spring of claim 5, wherein, on each end of the coiled spring, the outermost loop and the second from outermost loops of the coil are joined to one another by means of a welded connection in at least one point.

7. The coiled spring of claim 4, wherein the welded connection is a laser-welded connection.

8. The coiled spring of claim 4, wherein the coil is formed by a wire with a circular cross section.

9. The coiled spring of claim 8, wherein a diameter of the wire is less than 2 mm.

10. The coiled spring of claim 8, wherein the diameter of the wire is between 0.3 mm and 1.2 mm.

11. The coiled spring of claim 4, wherein the fixedly joined loops are joined together by laser welding at two separate points.

12. The coiled spring of claim 4, wherein the coiled spring comprises a coil that contains at least 5 loops.

13. The coiled spring of claim 4, wherein the coiled spring comprises a coil that contains between 6 and 30 loops.

14. The coiled spring of claim 4, wherein an external dimension of the coiled spring measured perpendicular to an axis of the spring is between 4 mm and 26 mm or between 9 mm and 21 mm.

15. The coiled spring of claim 14, wherein an external dimension of the coiled spring is at least 9 mm measured perpendicular to an axis of the spring, and the welded connection is at least 20 mm from an end of a wire forming the coiled spring.

16. A method for connecting two loops of a coiled spring for an injection device comprising the following steps:
provision of a coiled spring which extends as a coil in an axial direction with at least three loops of wire; and
connection of two adjacent loops in at least one point by welding.

17. The method of claim 16, wherein the welding is laser welding.

18. The method of claim 16, wherein the two adjacent loops are connected by laser welding at two separate points.

19. The method of claim 16, wherein the coiled spring has a first end and a second end, and two adjacent loops are connected at each of the first end and the second end.

20. The method of claim 16, wherein the connecting by welding is in an axial direction between adjacent loops.
